(19) United States
(12) Patent Application Publication
(10) Pub. No.: US 2007/0065850 A1
(43) Pub. Date: Mar. 22, 2007

(54) Method for diagnosis of heart failure

(75) Inventors: Tetuo Minamino, Suita-shi (JP); Masatsugu Hori, Suita-shi (JP); Ken-ichiro Okada, Suita-shi (JP); Masafumi Kitakaze, Suita-shi (JP)

Correspondence Address: SUGHRUE MION, PLLC, 2100 PENNSYLVANIA AVENUE, N.W., SUITE 800, WASHINGTON, DC, 20037, US

(73) Assignees: OSAKA UNIVERSITY; JAPAN HEALTH SCIENCES FOUNDATION

(21) Appl. No.: 11/501,810

(22) Filed: Aug. 10, 2006

(30) Foreign Application Priority Data
Sep. 20, 2005 (JP) 2005-272336
Jan. 25, 2006 (JP) 2006-016514

(51) Int. Cl. C12Q 1/68; G01N 33/567

(52) U.S. Cl. 435/6; 435/7.2

(57) ABSTRACT

A method for diagnosis of heart failure in a human subject, which comprises the step of: measuring an endoplasmic reticulum (ER)-initiated apoptotic signal such as CHOP in a biological sample originated from the subject is disclosed. According to the invention, by employing the new marker in stead of or in addition to a conventional cardiac marker such as BNP, more accurate diagnosis of heart failure can be attained.

### BACKGROUND OF THE INVENTION

### ART RELATED

The instant application relates to a method for diagnosis of heart failure in a human subject. The application also relates to a composition used for the diagnosis.

Diagnosis of heart failure as well as determination of the severity and prognosis of chronic heart failure have been performed using chest x-ray and ultrasound imaging in addition to the subjective and objective symptoms. Recently, brain natriuretic peptide (BNP), a cardiac-derived peptide hormone that circulates in the blood and exerts potent cardiovascular and renal actions, in the blood sample has been employed as a cardiac marker for more accurate diagnosis. The elevated plasma BNP level indicates a heart disease, for example, left ventricular dysfunction (8-10). However, the plasma BNP level may also be increased with aging, declining renal function, cardiac hypertrophy and the like. Therefore, diagnosis of heart failure based solely on BNP is not always accurate enough.

The endoplasmic reticulum (ER) is an organelle that participates in the folding of membrane proteins and secretory proteins (1-3). Various cellular stresses, including ischemia, hypoxia, heat shock, gene mutation, oxidative stress, and increased protein synthesis, lead to impaired functioning of the ER (1-3). Stimuli that cause ER dysfunction are collectively known as ER stress. To deal with ER stress, adaptive responses such as the up-regulation of ER-resident chaperones and the decline of translation that decreases new protein synthesis are induced (1-3). When ER stress is excessive and/or prolonged, apoptotic signals are issued from the ER (1, 3, 4), including induction of the transcription of C/EBP homologous protein (CHOP), activation of c-JUN NH2-terminal kinase (JNK), and cleavage of caspase-12 (1, 3-6).

C/EBP homologous protein (CHOP) is a protein belonging to the C/EBP family and consisting of 169 amino acids. This protein has been known as a specific signal mediating apoptotic cell death initiated by ER stress. The inventors have previously reported that expression of GRP78 and CHOP, both are ER-initiated apoptotic signals, were observed in failing human hearts(7). However, the role of ER stress and ER stress-induced apoptosis in the onset and progress of heart failure has not yet been revealed.

### SUMMARY OF THE INVENTION

An object of the invention is to provide a method as well as composition for diagnosis of heart failure.

The inventors have found that the amount of an ER-initiated apoptotic signal such as CHOP correlates with onset, severity and/or prognosis of heart failure and completed the invention.

In one aspect of the invention, a method for diagnosis of heart failure in a human subject, which comprises the step of: measuring an ER-initiated apoptotic signal in a biological sample originated from the patient is provided. The measurement may be carried out by determining the mRNA or protein amount of the signal in the sample. The sample may comprise cells obtained from the heart, especially, cardiac muscle cells. The preferably used ER-initiated apoptotic signals may include C/EBP homologous protein (CHOP). In the method of the invention, a cardiac marker other than an ER-initiated apoptotic signal in a biological sample originated from the subject may also be measured and the result may be used for the diagnosis in combination with the ER-initiated apoptotic signal.

In another aspect of the instant invention, a composition for diagnosis of heart failure in a human subject, which comprises a reagent for measuring an ER-initiated apoptotic signal in a biological sample originated from the subject. The composition may further comprise a reagent for measuring a cardiac marker other than an ER-initiated apoptotic signal in a biological sample originated from the subject.

Namely, the instant inventors have firstly found the followings:

1) In failing human heart, CHOP, an apoptotic cell death related transcription factor is induced by ER stress.

2) In failing human heart, expression of CHOP positively correlates with that of BNP. In this regard, significant correlation between plasma BNP level and BNP level in failing heart had previously been reported;

3) Pharmacological ER stress inducers increase the expression of CHOP and the number of apoptotic cell death in cultured neonatal rat cardiac myocytes;

4) ER stress-mediated apoptosis of cultured cardiac myocytes is attenuated by the posttranscriptional silencing of CHOP (RNA interference); and

5) In mice transverse aortic constriction (TAC) model, where progression of cardiac hypertrophy is induced at one week after TAC and the progression of cardiac hypertrophy to failing heart is observed at 4 weeks after TAC, expression of BNP is increased in both hypertrophic and failing hearts. Whereas, expression of CHOP is not increased in hypertrophic heart but increased only in failing heart.

Human plasma BNP has been used as a quantitative clinical marker for diagnosis of heart failure. Kits for determining plasma BNP such as Shionoria BNP (Shionogi Co., Ltd., Osaka, Japan) and Determiner-BNP (Kyowa Medex, Tokyo, Japan) are commercially available. The inventors have firstly found that a significant correlation between the expressions of BNP and CHOP is observed in failing human heart, and that the expression of CHOP is specifically induced in failing heart. Accordingly, the method of the application, i.e. that for diagnosis of heart failure in a subject which comprises the step of measuring an ER-initiated apoptotic signal in a biological sample originated from the patient, instead of, or in addition to measuring BNP in a biological sample originated from the subject, can provide more accurate diagnosis of heart failure as well as determination of severity and prognosis of chronic heart failure. Especially, the method of the invention is useful for determining the severity of the failing heart.

### BRIEF DESCRIPTION OF THE DRAWINGS

FIG. 1. Induction of ER stress and activation of ER-initiated apoptotic signaling in failing human hearts. Control 1 (a, e, i, m, r) and control 2 (b, f, j, n, s) are tissue samples obtained at autopsy from patients with leukemia and osteosarcoma, respectively. DCM 1 (c, g, k, o, t) and DCM 2 (d, h, l, p, u) are tissue samples obtained as surgical specimens from 2 patients with DCM. Immunohistochemical analysis of GRP78 (e-h) and CHOP (i-l) in human hearts. Scale bar represents 40 μm. In HE staining, no vacuolation in cardiac samples from control subjects (a, b) was observed. In contrast, vacuolation was prominent in the perinuclear region on examination of samples from failing human hearts (c, d). Importantly, localization of vacuolation was consistent with that of GRP78 or CHOP expression (g, h, k, l), suggesting that vacuolation indicated the dilation of ER lumen in cardiac myocytes. JNK was not phosphorylated in failing human hearts (o-p). Negative control sections using anti-IgG (anti-rabbit) serum as the primary antibody revealed no immunorcactivity in the nucleus, perinuclear area, and cytoplasm (r-u). The section in adenocarcinoma of human colon cancer was used as a positive control for anti-phospho-JNK serum (q).

FIG. 2. Expression of BNP, GRP78, and CHOP mRNA in failing human hearts. A. Plasma BNP levels in 13 patients with heart failure. The dotted line indicates the upper limit of the normal range for BNP (18.4 pg/mL). Plasma BNP levels (637.5±142.9 pg/mL) in all 13 patients were beyond the upper limit of the normal range. B. BNP mRNA expression quantitated by real-time RT-PCR was significantly (p<0.05) increased in the hearts of all 13 patients with DCM and ICM, while BNP mRNA expression was not detected in the hearts of 5 control subjects. C. Correlation between plasma BNP levels and cardiac mRNA levels of BNP in patients with chronic heart disease (CHF). There was a significant correlation between plasma BNP levels and cardiac mRNA levels of BNP (R=0.67, p<0.05). D. GRP78 mRNA expression quantitated by real-time RT-PCR was significantly (p<0.05) increased in the hearts of all 13 patients (3.4±0.6) compared with the 5 control subjects (1.0±0.1). E. CHOP mRNA expression quantitated by real-time RT-PCR was also significantly (p<0.05) increased in the hearts of all 13 patients (14.2±2.9) compared with the 5 control subjects (1.0±0.1). F, G, H. Correlations between the expression of BNP and ER stress markers. There was a positive correlation between expression of GRP78 and CHOP (r=0.68, P<0.05). There was also a positive correlation between expression of BNP and GRP78 (r=0.60, P<0.05) or CHOP (r=0.53, P<0.05). Results of real-time quantitative RT-PCR were normalized for GAPDH expression.

FIG. 3-1. Effect of CHOP gene interference on cardiac myocyte apoptosis induced by ER stress. A. Expression of CHOP after treatment of cultured neonatal rat cardiac myocytes with tunicamycin (Tu). Expression was quantitated by real-time RT-PCR. Results of real-time quantitative RT-PCR were normalized for GAPDH expression. * p<0.05 compared with 0 hour. B. Expression of CHOP after transfection of 3 different siRNA oligonucleotides. Real-time quantitative RT-PCR revealed that expression of CHOP was suppressed in tunicamycin-treated cardiac myocytes by all 3 siRNA oligonucleotides. C. Expression of CHOP protein after transfection of CHOP siRNA-2. In CHOP knockdown cardiac myocytes, expression of CHOP protein was almost completely suppressed after treatment with tunicamycin. Actin was used as an internal control. D. Representative results of the TUNEL assay in cultured cardiac myocytes after CHOP gene interference. Scale bar=20 μm.

E. Number of TUNEL-positive cells. Tunicamycin increased the number of TUNEL-positive cardiac myocytes, which was attenuated by CHOP siRNA-2, but not GL2 siRNA. * p<0.05 vs. no treatment (control). # p<0.05 vs. CHOP siRNA-2. F. Viability of cultured cardiac myocytes after treatment with tunicamycin. Tunicamycin decreased cell viability, which was attenuated by CHOP siRNA, but not GL2 siRNA. * p<0.05 vs. no treatment (control). # p<0.05 vs. CHOP siRNA-2.

FIG. 3-2. Enlarged vision of FIG. 3-1D.

FIG. 4. represents expression of GRP78, BNP and CHOP in hypertrophic hearts (1 week after TAC) and failing hearts (4 weeks after TAC). A: Expression of GRP78 mRNA in hypertrophic and failing hearts determined by Northern blotting analysis. The expression of GRP78 was significantly increased in the hypertrophic and failing hearts compared with the sham mice. B: Expression of CHOP protein in the hypertrophic and failing hearts determined by Western blotting analysis. Although CHOP was slightly observed in the hypertrophic hearts, the amounts were apparently increased in the failing hearts. C: Expression of BNP mRNA in the hypertrophic and failing hearts determined by the real time RT-PCR. The amounts of BNP were increased in both hypertrophic and failing hearts. *p<0.05 vs. sham mice.

### DETAILED DESCRIPTION OF THE PREFERRED EMBODIMENTS

According to the instant invention, “heart failure” diagnosed by the method of the invention may include acute myocardial infarction, acute heart failure, chronic heart failure, dilated cardiomyopathy, ischemic cardiomyopathy and cardiac valvular disease.

In the specification and claims, “endoplasmic reticulum-initiated apoptotic signal” or “ER-initiated apoptotic signal” represents any of signals initiated or generated by ER in the pathway wherein ER stress induces apoptosis. For example, the signal may be CHOP, caspase 12 and JNK. Among them, CHOP, is preferably used. It has been reported that CHOP is one of C/EBP family proteins and may form a heterodimer with a C/EBP family protein. In the specification and claims, the term “CHOP” also covers those C/EBP family proteins and the dimers.

In the instant specification and claims, “biological sample” may be that freshly obtained non-processed tissues or that prepared by processing said tissues obtained from the subject. The samples may be homogenate of the fresh tissue or pathological sections of the tissues.

According to the instant method, the absolute or relative amount of the ER-initiated apoptotic signal is determined and the heart failure is diagnosed based on the amount of the signal.

According to the instant invention, the subject to be diagnosed by the method is not limited and may be a patient susceptible to have failing heart based on clinical observations. The subject may also be the one with no clinical signs suggesting failing heart, for example, a person having his/her health checkup.

The biological sample originated from the subject is not limited and may be heart tissue comprising heart cells, especially, cardiac muscle cells.

The measurement of an ER-initiated apoptotic signal may be carried out by any method which can determine the absolute or relative amount of the signal in the sample. For example, the amount of protein or nucleic acid of the signal may be determined.

Examples of nucleic acids of the signal may include DNA and mRNA coding for the signal, and mRNA is preferably employed. The amount of mRNA in the sample may be determined by any of conventionally known procedures such as real time RT-PCR.

The protein amount of the signal in the sample may be determined by any of conventionally known analyses for protein amount determination such as Immunohistochemical analysis and immunoblotting.

As is discussed below, the inventors have firstly confirmed that the expression of CHOP, one of ER-initiated apoptotic signals, positively correlates with that of BNP in failing human hearts. BNP has been clinically used as a cardiac marker indicating the state of failing heart. Accordingly, the method of the invention comprising the measurement of an ER-initiated apoptotic signal in a biological sample originated from a subject can be employed not only for determining onset or development of failing heart, but also for assessing the cardiac function as well as state, severity and prognosis of failing heart. The term “diagnosis” or “diagnosing” in the specification and claims covers not only determining the onset of heart failure but also assessing the cardiac function, state, severity as well as prognosis of failing heart.

According to the instant invention, the obtained amount of the ER-initiated apoptotic signal may be used solely or in combination with the amount of a cardiac marker other than said signal in a sample originated from the subject in diagnosis of heart failure. The cardiac marker other than an ER-initiated apoptotic signal may be any of substances which can be used as markers indicating cardiac condition, including BNP, atrial natriuretic peptide (ANP), troponin T, troponin I, creatinine kinase MB (CK-MB) and heart type fatty acid binding protein (H-FABP). Among the markers, BNP is most preferably used in the method of the instant application.

The biological sample used for the measurement of the cardiac marker other than an ER-initiated apoptotic signal may be the same as or different from that used for the measurement of the ER-initiated apoptotic signal. In case a different biological sample is used for the cardiac marker other than an ER-initiated apoptotic signal, the biological sample may be blood sample such as whole blood, plasma or serum. The measurement of the other cardiac marker may be conducted according to any of procedures known to the art including clinically used protocols.

In more detail, the diagnosis of heart failure in a patient according to the method of the invention may comprise the steps as follows:

1) obtaining a biological sample from the subject,

2) measuring the amount of an ER-initiated apoptotic signal in the sample; and

3) comparing the obtained amount of the signal and a preliminarily prepared correlation curve or correlation table between the amount and a classification criterion for heart failure to diagnose heart failure in the subject.

In this regard, “classification criterion for heart failure” may be any of those used in this field and for example, a classification of patients with heart disease (based on the relation between symptoms and the amount of effort required to provoke them) developed by New York Heart Association may be employed. According to this embodiment, the correlation curve or table between the amount of the signal and the classification criteria is preliminarily prepared using samples obtained from plural subjects of various classes and then, the amount of the signal in the sample originated from the subject to be diagnosed is applied to the curve or table.

According to the instant application, a composition for diagnosis of heart failure in a human subject comprising a reagent for the measurement of an ER-initiated apoptotic signal in a biological sample originated from the subject is also provided. The reagent may be selected depending on the substance to be targeted, i.e. nucleic acid, protein and the like as well as the protocol used for the measurement. For example, in case the amount of DNA or mRNA is determined by means of real time RT-PCR, the composition may comprise primers and a probe. In case the protein amount of the signal is measured by means of immunoassay, immunohistochemical assay or immunoblotting assay, the composition may comprise a substance which specifically binds to the signal, such as an antibody specific to the ER-initiated apoptotic signal.

According to the instant application, the composition may further comprise a reagent for measuring a cardiac marker other than an ER-initiated apoptotic signal. The reagent may be selected from those conventionally or clinically used in the measurement of the marker.

The composition of the invention may be provided, reserved or distributed as a kit for diagnosing heart failure.

The instant invention will be understood more readily with the following examples. However, these examples are intended to illustrate the invention and are not to be construed to limit the scope of the invention.

Human heart samples were studied according to the protocol approved by the Institutional Review Boards of National Cardiovascular Center (Osaka, Japan No. 14-18) and Hayama Heart Center (Kanagawa, Japan). The heart samples for immunohistochemical analysis were obtained as surgical specimens from 2 patients with dilated cardiomyopathy (DCM) and at autopsy from 2 patients with acute leukemia and osteosarcoma (as a control). For real-time quantitative RT-PCR, the inventors used surgical samples of cardiac tissue from 10 patients with DCM and 3 patients with ischemic cardiomyopathy (ICM) who underwent left ventriculoplasty from October 2001 to December 2002 at Hayama Heart Center. Five control heart samples for real-time quantitative RT-PCR were commercially obtained from BD Biosciences. Tissue samples for extraction of RNA were frozen at −80° C. until use, while the specimens for immunohistochemistry were fixed and embedded in paraffin. The clinical characteristics of the 13 patients from Hayama Heart Center were evaluated just before they underwent left ventriculoplasty (Table 1).

TABLE 1

| | | | | | | Echocardiografic findings | | |
|---|---|---|---|---|---|---|---|---|
| | | Gen- | Diag- | BNP | NYHA | LVEF | LVDd | LVDs |
| | Age | der | nosis | (pg/mL) | class | (%) | (mm) | (mm) |
| 1 | 68 | M | ICM | 370 | IV | 36 | 100 | 90 |
| 2 | 50 | F | DCM | 417 | IV | 34 | 70 | 59 |
| 3 | 40 | M | DCM | 450 | III | 24 | 96 | 84 |
| 4 | 72 | M | DCM | 310 | III | 26 | 91 | 79 |
| 5 | 67 | M | DCM | 313 | III | 24 | 85 | 75 |
| 6 | 56 | M | ICM | 55 | III | 27 | 70 | 56 |
| 7 | 52 | M | DCM | 1711 | IV | 26 | 95 | 85 |
| 8 | 53 | M | DCM | 331 | III | 30 | 91 | 83 |
| 9 | 50 | M | DCM | 1610 | IV | 17 | 88 | 81 |
| 10 | 61 | M | DCM | 1030 | III | 11 | 81 | 77 |
| 11 | 48 | M | DCM | 800 | IV | 21 | 93 | 88 |
| 12 | 62 | M | DCM | 570 | III | 30 | 78 | 67 |
| 13 | 54 | M | ICM | 321 | III | 38 | 74 | 61 |

DCM, dilated cardiomyopathy;
ICM, ischemic cardiomyopathy;
BNP, brain-type natriuretic peptide;
NYHA, New York Heart Asociation function class;
LVEF, left ventrlicular ejection fraction;
LVDd, left ventricular end-diastolic dimension;
LVDs, left ventricular end-systolic dimension.

Preparation of Neonatal Rat Cardiac Myocytes

Primary cultures of cardiac myocytes were prepared from neonatal rat hearts as described previously (13). All procedures were performed in accordance with the guidline of Osaka University School of Medicine with regard to animal care and the “Position of the American Heart Association on Research Animal Use.”

Real-Time Quantitative RT-PCR

RT-PCR of human and rat heart samples was performed according to the Omniscript Reverse Transcription handbook (QIAGEN Inc.). The human and rat primers and probes used for quantification of BNP, GRP78, CHOP, and GAPDH were all designed according to the manufacturer's protocol (Applied Biosystems, https://www.appliedbiosystems.com/catalog/). Real-time quantitative RT-PCR was performed with an ABI PRISM 7700 Sequence Detection System (Applied Biosystems) by the relative standard curve method. The target amount was determined from the relative standard curves constructed with serial dilutions of the control total RNA.

Immunohistochemical Analysis

Immunohistochemical analysis was performed as described previously (14). Primary antibodies targeting GRP78 (sc-1050) and CHOP (sc-793) were purchased from Santa Cruz Biotechnology Inc. Phospho-JNK antibody was purchased from Cell Signaling Technology, Inc. The tissue section slide of adenocarcinoma of human colon cancer was purchased from BioChain Institute, Inc.

Immunoblotting

Immunoblotting was performed as described previously (13). Primary antibodies targeting CHOP (sc-793) and actin (sc-1615) were purchased from Santa Cruz Biotechnology. Forty eight hours after the treatment of neonatal rat cardiac myocytes with tunicamycin (Sigma-Aldrich), cardiac myocytes were harvested.

Apoptotic Cell Assay

The terminal deoxynucleotidyl transferase-mediated dUTP nick end labeling (TUNEL) assay was performed as described previously (7). Forty eight hours after the treatment of neonatal rat cardiac myocytes with tunicamycin (Sigma-Aldrich), cells were subjected to TUNEL staining. The TUNEL-positive cardiac myocytes counted in DAPI-positive cardiac myocytes (n=100, 5 times) were expressed as a percentage.

MTT Assay

Cardiac myocytes were treated with tunicamycin for 48 hours, after which cell viability was assessed by MTT assay according to the manufacturer's instructions (Cell Counting Kit-8, Wako Pure Chemical Industries, Ltd.). The percentage of viable cells was determined by taking the value for untreated cells as 100%.

RNA Interference (RNAi)

The inventors designed 3 different short interfering RNAs (siRNA) with 3′ dTdT overhangs to knock down CHOP mRNA (CHOP siRNA-1: 5′-CCUUCACUACUCUUGACCC-3′ (SEQ ID NO. 1), siRNA-2: 5′-GAUCAAGGAAGAACUAGGA-3′ (SEQ ID NO. 2), siRNA-3: 5′-GGAGCAGGAGAAUGAGAGG-3′(SEQ ID NO. 3) and ordered Dharmacon Inc. to synthesize them. After rat cardiac myocytes were isolated, they were incubated in Dulbecco's modified Eagle's medium (Invitrogen Corporation) Opti-MEM (Invitrogen Corporation), siRNA oligonucleotides (CHOP siRNA 1-3) (60 nM) and Optifect (Invitrogen Corporation) were added on the day of cardiac myocyte isolation. As a negative control, cells were transfected with siRNA against firefly luciferase from Photinus pyralis (GL2 siRNA) (15). Messenger RNA and protein levels of CHOP were measured 24 and 48 hrs after the transfection of CHOP siRNA, respectively.

Mice Transverse Aortic Constriction (TAC) Model

C57BL/6 mice (aged 8 weeks; male) were subjected to transverse aortic constriction (TAC) or sham operation as described previously(28). By echocardiographic analysis, development of cardiac hypertrophy was observed at 1 week after TAC and the condition progressed to failing heart at 4 weeks after TAC. GRP78 mRNA level, CHOP protein level and BNP mRNA level in the hearts of the mice at 1 and 4 weeks after TAC were measured by means of northern blotting, western blotting and RT-PCR, respectively.

Statistical Analysis

Data are expressed as the mean±SEM. The unpaired t-test was used to compare the mRNA levels of BNP, those of GRP78, and those of CHOP in control group and failing human hearts group, separately. Pearson's correlation coefficient analysis was used to examine the relationship between the mRNA levels of BNP and those of GRP78 as well as the relationship between the mRNA levels of BNP and those of CHOP. The results of the RNAi method using cultured rat cardiac myocytes were compared by one-way factorial ANOVA followed by Bonferroni's correction. For all analyses, P<0.05 was accepted as statistically significant.

Results

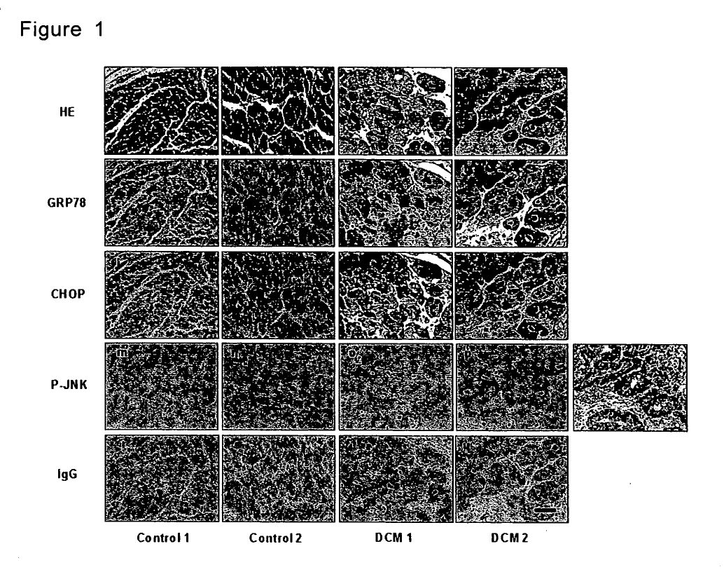

The morphological development of the ER and increased expression of GRP78 and CHOP in failing human hearts Examination of hematoxylin-eosin (HE) stained sections showed no vacuolation in cardiac samples from control subjects (FIGS. 1a, b). In contrast, vacuolation was prominent in the perinuclear region on examination of samples from failing human hearts (FIGS. 1c, d). Immunohistochemical analysis revealed that GRP78 expression was increased in the cardiac samples from patients with DCM (FIGS. 1g, h), but not from control subjects (FIGS. 1e, f). CHOP expression was also increased in cardiac samples from patients with DCM (FIGS. 1k, l), but not control subjects (FIGS. 1i, j). Importantly, localization of vacuolation in HE staining was consistent with that of GRP78, an ER-resident chaperone, suggesting that vacuolation in failing hearts indicated the dilatation of ER lumen in cardiac myocytes. In contrast, JNK was not phosphorylated in patients with heart failure (FIGS. 1o, p), suggesting that in failing human hearts, CHOP is induced, but JNK is not induced to a transfer system of apoptotic signal issued from ER. The section in adenocarcinoma of human colon cancer was used as a positive control for anti-phospho-JNK antibody (q). Negative control sections using anti-IgG serum instead of anti-CHOP as the primary antibody revealed no immunoreactivity in the nucleus, perinuclear area, and cytoplasm (FIGS. 1r-u). When anti-IgG (anti-goat) serum was used instead of anti-GRP78 as the primary antibody, there was also no immunoreactivity (data not shown). The morphological changes of the ER and the increased expression of GRP78 and CHOP suggested that ER stress was induced and ER stress-mediated apoptotic signaling was activated in failing human hearts.

Positive Correlation between Expression of ER Stress Markers and BNP in Failing Human Hearts

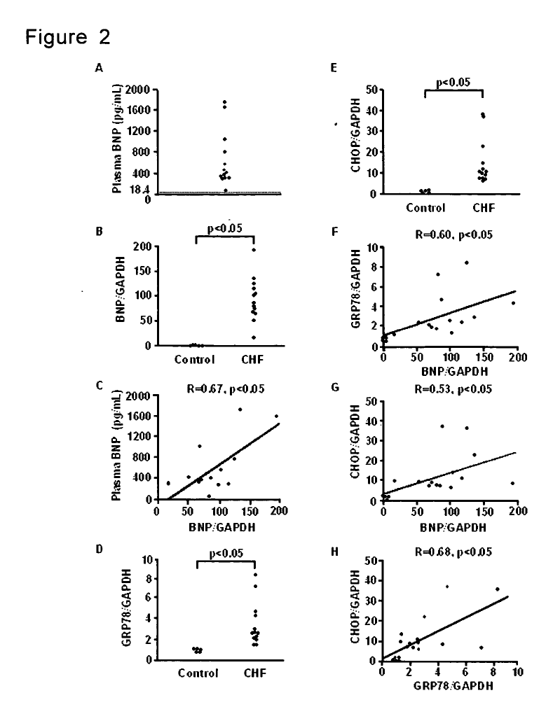

The clinical characteristics of the patients providing tissue specimens are shown in Table 1. Plasma BNP levels (637.5±142.9 pg/mL) of all 13 patients were markedly beyond the upper limit of the normal range (FIG. 2A). Since it was difficult to obtain enough human heart samples for repeated performance of Northern blotting, the inventors used real-time quantitative RT-PCR to evaluate the relationship between ER stress markers and BNP. Expression of BNP was markedly increased in the hearts of all 13 patients with DCM or ICM, while BNP expression was not detected in the hearts of the 5 control subjects (FIG. 2B). There was a significant correlation between the plasma BNP level and the cardiac level of BNP mRNA (R=0.67, p<0.05, FIG. 2C) in the patients with CHF, suggesting that plasma BNP reflects the extent of BNP production in failing human hearts.

GRP78 and CHOP expression were also examined by real-time quantitative RT-PCR. GRP78 expression was significantly (p<0.05) increased in the hearts of all 13 patients (GRP78/GAPDH: 3.4±0.6) compared with the 5 control subjects (GRP78/GAPDH: 1.0±0.1) (FIG. 2D). CHOP expression was also significantly (p<0.05) increased in the hearts of all 13 patients (CHOP/GAPDH: 14.2±2.9) compared with the 5 control subjects (CHOP/GAPDH: 1.0±0.1) (FIG. 2E). Furthermore, the inventors found a significant correlation between GRP78 and CHOP expression (r=0.68, P<0.05) (FIG. 2F). Interestingly, there was a positive correlation between expression of BNP and that of GRP78 (r=0.60, P<0.05) and a positive correlation between expression of BNP and that of CHOP (r=0.53, P<0.05) in failing human hearts. These findings suggest that ER stress and ER-initiated apoptotic signal are augmented in failing human hearts as cardiac dysfunction progressed.

Attenuation of cardiac myocyte apoptosis, which is caused by ER stress, by posttranslational silencing of CHOP gene

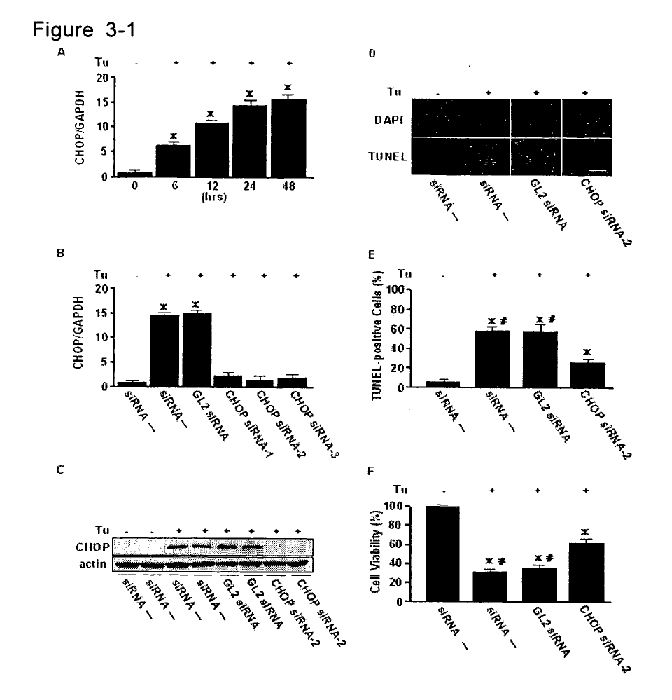

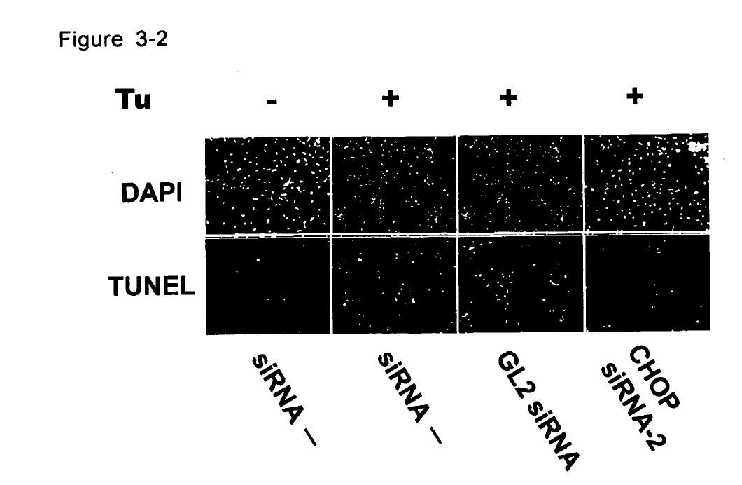

To determine whether a pharmacological ER stress inducer leads to increased expression of CHOP in hearts, which was up-regulated in failing human hearts, the inventors examined the expression of CHOP in cultured neonatal rat cardiac myocytes treated with tunicamycin, a potent ER stress inducer (1, 4, 5). Real-time quantitative RT-PCR revealed that tunicamycin caused an increase of CHOP expression in cultured cardiac myocytes (FIG. 3A). The expression of CHOP was also significantly (p<0.05) increased in cultured cardiac myocytes 48 hours after treatment with other pharmacological ER stress inducers such as thapsigargin (CHOP/GAPDH: 1.0±0.1 vs. 19.0±0.3) or brefeldin A (CHOP/GAPDH: 1.0±0.3 vs. 8.1±0.7).

To determine the role of CHOP in ER stress-induced apoptosis of cardiac myocytes, the inventors used siRNA oligonucleotides for posttranslational silencing of the CHOP gene. First, the inventors examined the mRNA level of CHOP after transfection of 3 different siRNAs against CHOP. All 3 siRNA oligonucleotides significantly reduced the mRNA level of CHOP (FIG. 3B). The inventors also confirmed that transfection of CHOP siRNA-2 significantly (p<0.05) attenuated the induction of CHOP expression by thapsigargin (CHOP/GAPDH: 19.0±0.3 vs. 2.1±1.0) or brefeldin A (CHOP/GAPDH: 8.1±0.7 vs. 2.2±0.8). Furthermore, the inventors found that the protein level of CHOP was almost completely suppressed by the transfection of CHOP siRNA-2 into cardiac myocytes (FIG. 3C). Tunicamycin increased the number of TUNEL-positive cardiac myocytes, which was significantly (p<0.05) attenuated by siRNA oligonucleotides against CHOP, but not attenuated by siRNA oligonucleotides against GL2 (FIGS. 3D, E). In addition, tunicamycin decreased cell viability, which was significantly (p<0.05) attenuated by siRNA oligonucleotides against CHOP, but not attenuated by siRNA oligonucleotides against GL2 (FIG. 3F). These findings suggest that ER stress can induce cardiac apoptosis via a CHOP-dependent pathway.

Mice Tac Model

One week after the onset of TAC, cardiac enlargement was detected without severe lung congestion. In contrast, cardiac enlargement was more prominent along with marked lung congestion 4 weeks after TAC. Echocardiographic analysis also revealed left ventricular (LV) dilatation and LV systolic dysfunction 4 weeks, but not 1 week, after TAC. Increases in LV wall thickness were found 1 week after TAC and thereafter. These findings indicate that the mice 1 and 4 weeks after surgery corresponded to models of cardiac hypertrophy and failure, respectively.

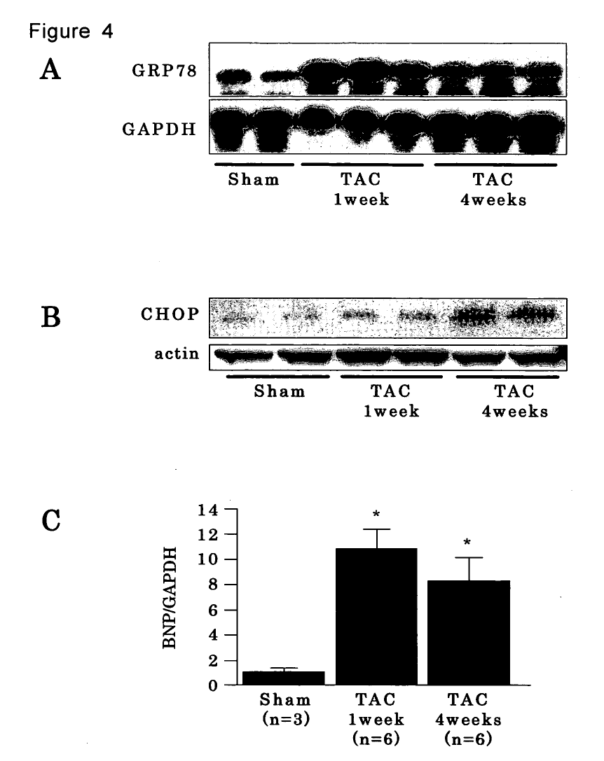

Expression of GRP78, BNP and CHOP in the heart of the mice 1 and 4 weeks after TAC were determined. Cardiac expressions of GRP78 and BNP were significantly increased at 1 and 4 weeks after TAC compared with the sham mice. Cardiac expression of CHOP at 1 week after TAC was substantially the same as of the control but that was significantly increased at 4 weeks compared with the sham mice (FIG. 4). This result indicates expression of CHOP is specifically increased in failing heart.

### REFERENCES

The following references are herein incorporated by reference.
1. Kaufman R J. Stress signaling from the lumen of the endoplasmic reticulum: coordination of gene transcriptional and translational controls. Genes Dev. 1999;13:1211-1233.
2. Rutkowski D T, Kaufman R J. A trip to the ER: coping with stress. Trends Cell Biol. 2004;14:20-28.
3. Kaufman R J. Orchestrating the unfolded protein response in health and disease. J Clin Invest. 2002;110:1389-1398.
4. Breckenridge D G, Germain M, Mathai J P, Nguyen M, Shore G C. Regulation of apoptosis by endoplasmic reticulum pathways. Oncogene. 2003;22:8608-8618.
5. Zinszner H, Kuroda M, Wang X, Batchvarova N, Lightfoot R T, Remotti H, Stevens J L, Ron D. CHOP is implicated in programmed cell death in response to impaired function of the endoplasmic reticulum. Genes Dev. 1998;12:982-995.
6. Oyadomari S, Mori M. Roles of CHOP in endoplasmic reticulum stress. Cell Death Differ. 2004;11:381-389.
7. Okada K, Minamino T. Tsukamoto Y, Liao Y, Tsukamoto O, Takashima S, Hirata A, Fujita M, Nagamachi Y, Nakatani T, Yutani C, Ozawa K, Ogawa S, Tomoike H, Hori M, Kitakaze M. Prolonged endoplasmic reticulum stress in hypertrophic and failing heart after aortic constriction: possible contribution of endoplasmic reticulum stress to cardiac myocyte apoptosis. Circulation. 2004;110:705-712.
8. Tsutamoto T, Wada A, Maeda K, Hisanaga T, Maeda Y, Fukai D, Ohnishi M, Sugimoto Y, Kinoshita M. Attenuation of compensation of endogenous cardiac natriuretic peptide system in chronic heart failure: prognostic role of plasma brain natriuretic peptide concentration in patients with chronic symptomatic left ventricular dysfunction. Circulation. 1997;96:509-516.
9. McDonagh T A, Robb S D, Murdoch D R, Morton J J, Ford I, Morrison C E, Tunstall-Pedoe H, McMurray J J, Dargie H J. Biochemical detection of left-ventricular systolic dysfunction. Lancet. 1998;351:9-13.
10. Lubien E, DeMaria A, Krishnaswamy P, Clopton P, Koon J, Kazanegra R, Gardetto N, Wanner E, Maisel A S. Utility of B-natriuretic peptide in detecting diastolic dysfunction: comparison with Doppler velocity recordings. Circulation. 2002;105:595-601.
11. Wencker D, Chandra M, Nguyen K, Miao W, Garantziotis S, Factor S M, Shirani J, Armstrong R C, Kitsis R N. A mechanistic role for cardiac myocyte apoptosis in heart failure. J Clin Invest. 2003;111:1497-1504.
12. Hannon G J. RNA interference. Nature. 2002;410:244-251.
13. Minamino T, Gaussin V, DeMayo F J, Schneider M D. Inducible gene targeting in postnatal myocardium by cardiac-specific expression of a hormone-activated Cre fusion protein. Circ Res. 2001;88:587-592.
14. Minamino T, Yujiri T, Terada N, Taffet G E, Michael L H, Johnson G L, Schneider M D. MEKK1 is essential for cardiac hypertrophy and dysfunction induced by Gq. Proc Natl Acad Sci U S A. 2002;99:3866-3871.
15. Elbashir S M, Harborth J, Lendeckel W, Yalcin A, Weber K, Tuschl T. Duplexes of 21-nucleotide RNAs mediate RNA interference in cultured mammalian cells. Nature. 2001;411:494-498.
16. Flesch M, Margulies K B, Mochmann H C, Engel D, Sivasubramanian N, Mann D L. Differential regulation of mitogen-activated protein kinases in the failing human heart in response to mechanical unloading. Circulation. 2001;104:2273-2276.
17. Ng D C, Court N W, dos Remedios C G, Bogoyevitch M A. Activation of signal transducer and activator of transcription (STAT) pathways in failing human hearts. Cardiovasc Res. 2003;57:333-346.
18. Hitomi J, Katayama T, Eguchi Y, Kudo T, Taniguchi M, Koyama Y, Manabe T, Yamagishi S, Bando Y, imaizumi K, Tsujimoto Y, Tohyama M. Involvement of caspase-4 in endoplasmic reticulum stress-induced apoptosis and A beta-induced cell death. J Cell Biol. 2004;165:347-356.
19. Mizuno Y, Yoshimura M, Harada E, Nakayama M, Sakamoto T, Shimasaki Y, Ogawa H, Kugiyama K, Saito Y, Nakao K, Yasue H. Plasma levels of A- and B-type natriuretic peptides in patients with hypertrophic cardiomyopathy or idiopathic dilated cardiomyopathy. Am J Cardiol. 2000;86:1036-1040.
20. Wiese S, Breyer T, Dragu A, Wakili R, Burkard T, Schmidt-Schweda S, Fuchtbauer E M, Dohrmann U, Beyersdorf F, Radicke D, Holubarsch C J. Gene expression of brain natriuretic peptide in isolated atrial and ventricular human myocardium: influence of angiotensin II and diastolic fiber length. Circulation. 2000;102:3074-3079.
21. Luodonpaa M, Vuolteenaho O, Eskelinen S, Ruskoaho H. Effects of adrenomedullin on hypertrophic responses induced by angiotensin II, endothelin-1 and phenylephrine. Peptides. 2001;22:1859-1866.
22. Goetze J P, Christoffersen C, Perko M, Arendrup H, Rehfeld J F, Kastrup J, Nielsen L B. Increased cardiac BNP expression associated with myocardial ischemia. FASEB J. 2003;17:1105-1107.
23. Kjaer A, Hesse B. Heart failure and neuroendocrine activation; diagnostic, prognostic and therapeutic perspectives. Clin Physiol. 2001;21:661-672.
24. Bettencourt P, Frioes F, Azevedo A, Dias P, Pimenta J, Rocha-Goncalves F, Ferreira A. Prognostic information provided by serial measurements of brain natriuretic peptide in heart failure. Int J Cardiol. 2004;93:45-48.
25. Shen C, Buck A K, Liu X, Winkler M, Reske S N. Gene silencing by adenovirus-delivered siRNA. FEBS Lett. 2003;539:111-114.
26. Jain K K. RNAi and siRNA in target validation. Drug Discov Today 2004;9:307-309.
27. Juhaszova M, Zorov D B, Kim S H, Pepe S, Fu Q, Fishbein K W, Ziman B D, Wang S, Ytrehus K, Antos C L, Olson E N, Sollott S J. Glycogen synthase kinase-3beta mediates convergence of protection signaling to inhibit the mitochondrial permeability transition pore. J Clin Invest. 2004;113:1535-1549.
28. Liao Y, Ishikura F, Beppu S, et al. Echocardiographic assessment of LV hypertrophy and function in aortic-banded mice: necropsy validation. Am J Physiol. 2002; 282: 1703-1708.

What is claimed is:

1. A method for diagnosis of heart failure in a human subject, which comprises the step of: measuring an endoplasmic reticulum (ER)-initiated apoptotic signal in a biological sample originated from the patient.

2. The method of claim 1, further comprising the step of: measuring a cardiac marker other than an ER-initiated apoptotic signal in a biological sample originated from the subject.

3. The method of claim 1, wherein the measurement of ER-initiated apoptotic signal is carried out by determining the amount of mRNA or protein of the signal.

4. The method of claim 1, wherein the biological sample is a sample comprising cells derived from the heart.

5. The method of claim 4, wherein the cells derived from the heart are cardiac muscle cells.

6. The method of claim 5, wherein the ER-initiated apoptotic signal is C/EBP homologous protein.

7. The method of claim 2, wherein the cardiac marker other than an ER-initiated apoptotic signal is brain natriuretic peptide (BNP).

8. The method of claim 2, wherein the ER-initiated apoptotic signal is C/EBP homologous protein and the cardiac marker other than an ER-initiated apoptotic signal is BNP.

9. A composition for diagnosis of heart failure in a human subject, which comprises a reagent for measuring an ER-initiated apoptotic signal in a biological sample originated from the subject.

10. The composition of claim 9, further comprising a reagent for measuring a cardiac marker other than an ER-initiated apoptotic signal in a biological sample originated from the subject.

11. The composition of claim 9, wherein the reagent for measuring an ER-initiated apoptotic signal is selected from the group consisting of that for measuring the amount of mRNA of the signal and that for measuring the amount of the protein of the signal.

12. The composition of claim 10, wherein the reagent for measuring an ER-initiated apoptotic signal is selected from the group consisting of that for measuring the amount of mRNA of the signal and that for measuring the amount of the protein of the signal.

13. The composition of claim 9, wherein the ER-initiated apoptotic signal is C/EBP homologous protein.

14. The composition of claim 10, wherein the cardiac marker other than an ER-initiated apoptotic signal is BNP.

15. The composition of claim 10, wherein the ER-initiated apoptotic signal is C/EBP homologous protein and the cardiac marker other than an ER-initiated apoptotic signal is brain natoriureic peptide.


SEQUENCE LISTING (3 sequences)

SEQ ID NO: 1 - RNA, 19 nt, Artificial
  short interfering RNA
ccuucacuacucuugaccc

SEQ ID NO: 2 - RNA, 19 nt, Artificial
  short interfering RNA
gaucaaggaagaacuagga

SEQ ID NO: 3 - RNA, 19 nt, Artificial
  short interfering RNA
ggagcaggagaaugagagg